(19) United States
(12) Patent Application Publication
(10) Pub. No.: US 2026/0157505 A1
(43) Pub. Date: Jun. 11, 2026

(54) DEVICE AND METHOD FOR MANUFACTURING USER-CUSTOMIZED HOME BEAUTY MASK PACK

(71) Applicant: SKINDAUM CO., LTD, Hwaseong-si, Gyeonggi-do (KR)

(72) Inventor: Young Kyoung LEE, Seoul (KR)

(21) Appl. No.: 19/126,981

(22) PCT Filed: Nov. 1, 2023
(86) PCT No.: PCT/KR2023/017223

§ 371 Date: May 2, 2025

(30) Foreign Application Priority Data
Nov. 4, 2022 (KR) 10-2022-0146021
Oct. 24, 2023 (KR) 10-2023-0142930

(51) Int. Cl. A45D 44/00; A61K 8/02

(52) U.S. Cl. CPC A45D 44/002; A61K 8/0212

(57) ABSTRACT

Proposed are a device and a method for manufacturing a user-customized home beauty mask pack. The device includes a mask pack sheet supply unit for separately accommodating various types of mask pack sheets and including a mask pack sheet outlet port, a raw material supply unit for separately accommodating a plurality of functional raw materials provided as a beauty and nutritional ingredient of a mask pack and including a functional raw material spout port, a control unit for controlling the mask pack sheet supply unit and the raw material supply unit so that one of the mask pack sheets is discharged through the outlet port and the functional raw materials are ejected through the spout port according to a mixing ratio, and a mask pack post-processing unit which accommodates the discharged mask pack sheet and the ejected functional raw materials in the internal space thereof.

### TECHNICAL FIELD

The present disclosure relates to skincare technology and, more particularly, to a device and method for manufacturing a user-customized home beauty mask pack.

### BACKGROUND ART

With aging, human skin undergoes both physical and chemical changes such as loss of skin elasticity, formation of wrinkles, changes in skin color, loss of skin barrier function, and loss of skin immune function. In particular, external environmental irritants like heavy metals, yellow dust, ozone, ultraviolet rays, and certain cosmetic ingredients promote decreased skin cell activity, necrosis (cell death), and cell degeneration, leading to wrinkle formation, pigmentation, loss of immune function, and increased inflammation. Beauty mask packs can help prevent and alleviate these issues. Conventional beauty mask packs have been manufactured to offer benefits like whitening (brightening), wrinkle treatment, moisturizing, skin trouble relief, and skin elasticity as the masks are often infused with essences containing nutrients such as natural extracts, proteins, and vitamins on woven or non-woven fabrics.

However, conventional beauty mask packs are not optimized for consumers but are manufactured from the producer's perspective even though each individual exhibits unique skin characteristics due to variations in factors like skin color, aging level, sensitivity, genetic makeup, etc., and the locations and areas of the eyes, nose, and mouth are different. Thus, the effectiveness of mask packs varies, and as the mask packs do not accurately reflect the user's individual face size and skin needs by area, the effective ingredients are distributed throughout the entire mask pack and cannot be applied to the desired area, and the effectiveness may be reduced due to the loss of effective ingredients.

Recently, the home-use beauty device market is booming as the number of people who practice skincare and haircare at home is increasing. As the novel coronavirus infection (COVID-19) is prolonged, the number of consumers who want to use beauty devices at home for easy and convenient management instead of visiting skin care facilities or dermatologists is rising. In addition, in line with the market situation, beauty-related products, which were limited to LED masks and cleansing devices, are becoming increasingly specialized, and various skin care devices that incorporate IT technology are being released.

The problem is that, because these home-use beauty devices are based on passive input information from users, the devices have limitations in providing genuinely personalized beauty services that are tailored to individual's specific skin concerns by area or type, etc.

### DISCLOSURE

### Technical Problem

An objective of the present disclosure is to provide a device for manufacturing a personalized home beauty mask pack.

In addition, an objective of the present disclosure is to provide a device for manufacturing a user-customized home beauty mask pack, the device boasting simplicity, convenience, cost-effectiveness, and efficiency, and enabling precise skin diagnosis.

In addition, another objective of the present disclosure is to provide a method for manufacturing a user-customized home beauty mask pack having the aforementioned advantages.

### Technical Solution

According to an embodiment of the present disclosure, there is provided a device for manufacturing a user-customized home beauty mask pack, the device including: a mask pack sheet supply unit configured to separately accommodate various types of mask pack sheets and include a mask pack sheet outlet port; a functional raw material supply unit configured to separately accommodate a plurality of functional raw materials provided as a beauty and nutritional ingredient of a mask pack and include a functional raw material spout port; a control unit configured to control the mask pack sheet supply unit and the functional raw material supply unit so that one of the mask pack sheets is discharged through the mask pack sheet outlet port and the functional raw materials are ejected through the functional raw material spout port according to a mixing ratio; and a mask pack post-processing unit configured to include an internal space open in a direction facing a discharge direction of the mask pack sheet outlet port and an ejection direction of the functional raw material spout port, and accommodate the discharged mask pack sheet and the ejected functional raw materials in the internal space so that the ejected functional raw materials are absorbed into the discharged mask pack sheet.

In an embodiment, the control unit may receive user skin diagnosis information by being connected to a user terminal or at least one server through a communication unit to determine the one of the mask pack sheets and the mixing ratio of the functional raw materials, may control the mask pack sheet outlet port so that the determined mask pack sheet is discharged from the mask pack sheet supply unit based on the user skin diagnosis information, and may control the functional raw material spout port so that the functional raw materials are ejected from the functional raw material supply unit according to the determined mixing ratio. The mask pack sheet supply unit may include: at least one mask pack sheet accommodation unit configured to accommodate mask pack sheets in a stacked manner according to a type of mask pack; and at least one mask pack sheet outlet unit configured to discharge, according to a control signal of the control unit, a lowermost mask pack sheet among mask pack sheets stacked in a corresponding mask pack sheet accommodation unit to the mask pack post-processing unit. The functional raw material supply unit may include: at least one functional raw material accommodation unit configured to accommodate functional raw materials respectively corresponding to a moisturizing ingredient, a skin-drying prevention ingredient, a whitening ingredient, and an elasticity ingredient; and at least one functional raw material discharge unit configured to eject, according to a control signal of the control unit, the functional raw materials accommodated in the functional raw material accommodation unit to the mask pack post-processing unit.

In an embodiment, the functional raw material supply unit may further include a mixing member configured to mix a plurality of functional raw materials supplied according to the mixing ratio from the functional raw material accommodation unit and deliver the mixed functional raw materials to the functional raw material discharge unit. The mask pack post-processing unit may include a Peltier thermoelectric element for cooling or heating a mask pack sheet and functional raw materials in the internal space through a heat absorption process or a heat generation process according to a control signal of the control unit. The device may further include a rotary member configured to rotate, at a first time point, the mask pack sheet supply unit so that the discharge direction of a mask pack sheet of the mask pack sheet outlet port and the opening direction of the mask pack post-processing unit are aligned, and rotate, at a second time point, different from the first time point, the mask pack post-processing unit so that the opening direction of the mask pack post-processing unit and the ejection direction of functional raw materials of the functional raw material discharge unit are aligned. The device may further include a transfer member configured to place, at a first time point, the mask pack post-processing unit in a first position so that the discharge direction of a mask pack sheet of the mask pack sheet outlet port and the opening direction of the mask pack post-processing unit are aligned, and transfer, at a second time point, different from the first time point, the mask pack post-processing unit from the first position to a second position so that the opening direction of the mask pack post-processing unit and the ejection direction of functional raw materials of the functional raw material discharge unit are aligned.

In an embodiment, the control unit may provide a user with information about a shape of the mask pack sheet to be discharged and the functional raw materials to be absorbed into the mask pack sheet before discharging the mask pack sheet into the internal space of the mask pack post-processing unit or before ejecting the functional raw materials into the internal space of the mask pack post-processing unit. The control unit may correct the shape of the mask pack sheet and a mixing ratio of the functional raw materials to be absorbed into the mask pack sheet in response to a response signal from the user, and may control a discharge and ejection of the mask pack sheet and the functional raw materials into the internal space of the mask pack post-processing unit according to the corrected shape of the mask pack sheet and the corrected mixing ratio of the functional raw materials. The types of the mask pack sheets may include any one of the following: U-zone, T-zone, nose pack, eye pack, eye wrinkle pack, neck pack, and face mask.

According to another embodiment of the present disclosure, there is provided a method for manufacturing a user-customized home beauty mask pack, the method including: separately accommodating various types of mask pack sheets in a mask pack sheet supply unit including a mask pack sheet outlet port; separately accommodating a plurality of functional raw materials provided as a beauty and nutritional ingredient of a mask pack in a raw material supply unit including a functional raw material spout port; discharging one of the mask pack sheets to the mask pack sheet supply unit through the mask pack sheet outlet port according to a control signal from a control unit; controlling the raw material supply unit so that the functional raw materials are ejected according to a mixing ratio through the functional raw material spout port; and discharging the one of the mask pack sheets into an internal space of a mask pack post-processing unit that is open in a direction facing a discharge direction of the mask pack sheet outlet port and an ejection direction of the functional raw material spout port, and ejecting functional raw materials onto the mask pack sheet according to the mixing ratio.

In an embodiment, the method may further include receiving user skin diagnosis information by being connected to a user terminal or at least one server through a communication unit to determine the one of the mask pack sheets and the mixing ratio of the functional raw materials, controlling the mask pack sheet outlet port so that the determined mask pack sheet is discharged from the mask pack sheet supply unit based on the user skin diagnosis information, and controlling the functional raw material spout port so that the functional raw materials are ejected from the raw material supply unit according to the determined mixing ratio. The skin diagnosis information may include skin's elasticity, moisture, pigmentation, anti-oxidation, and sensitivity levels, the skin's elasticity, moisture, pigmentation, anti-oxidation, and sensitivity levels may be predicted from data including user's pores, skin temperature, oil, moisture, skin tone, skin pH, skin thickness, wrinkles, pigmentation, blemishes, sebum, and dead skin cells. The skin diagnosis information may further include skin measurement information, and the skin measurement information may include at least one of a degree of UV exposure for a user, oil and moisture content in user's skin, skin temperature, skin pH, and skin thickness of the user.

According to still another embodiment of the present disclosure, there is provided a device for manufacturing a user-customized home beauty mask pack, the device including: a mask pack post-processing unit configured to place a capsule-type mask pack product into an internal space thereof, wherein the capsule-type mask pack product consists of a mask pack sheet, a container for accommodating the mask pack sheet, and a cover sheet for sealing the container; a functional raw material supply unit including at least one cartridge configured to separately accommodate a plurality of functional raw materials provided as a beauty and nutritional ingredient of a mask pack, and at least one puncture nozzle integrally connected with the cartridge; and a control unit configured to lower the puncture nozzle integrally connected with the cartridge to puncture the cover sheet of the capsule-type mask pack product, control an end of the puncture nozzle to enter the container of the capsule-type mask pack product, and control an injection of the functional raw materials into the container of the capsule-type mask pack product according to a mixing ratio so that the functional raw materials are absorbed into the mask pack sheet.

### Advantageous Effects

According to an embodiment of the present disclosure, a device for manufacturing a user-customized home beauty mask pack can be provided that is simple, convenient, economical, and efficient and allows users to produce and use mask packs at home by including: a mask pack sheet supply unit for accommodating various types of mask pack sheets; a functional raw material supply unit for accommodating a plurality of functional raw materials; a control unit for controlling the mask pack sheet supply unit and the raw material supply unit so that one of the mask pack sheets is discharged and the functional raw materials are ejected according to a mixing ratio; and a mask pack post-processing unit which accommodates the discharged mask pack sheet and the ejected functional raw materials in the internal space thereof.

In addition, according to another embodiment of the present disclosure, a method for manufacturing a user-customized home beauty mask pack capable of precise skin diagnosis with the above advantages can be provided.

However, the effects of the present disclosure are not limited to the above ones, and can be expanded in various ways without departing from the technical idea and scope of the present disclosure.

### DESCRIPTION OF DRAWINGS

FIG. 1 is a configuration view of a system for manufacturing a user-customized home beauty mask pack according to an embodiment of the present disclosure.

FIG. 2 is a configuration view of a device for manufacturing a user-customized home beauty mask pack according to an embodiment of the present disclosure.

FIGS. 3A to 3D are detailed views of a mask pack sheet supply unit according to an embodiment of the present disclosure.

FIGS. 4A and 4B are detailed views of a functional raw material supply unit according to an embodiment of the present disclosure.

FIG. 5 is a detailed view of a mask pack post-processing unit according to an embodiment of the present disclosure.

FIGS. 6A and 6B are views showing the discharge of a mask pack sheet and the ejection of a functional raw material according to an embodiment of the present disclosure.

FIGS. 7A and 7B are views showing the discharge of a mask pack sheet and the ejection of a functional raw material according to another embodiment of the present disclosure.

FIGS. 8A and 8B are flowcharts for showing a method for manufacturing a user-customized home beauty mask pack according to an embodiment of the present disclosure.

FIG. 9 is an example of a capsule-type mask pack sheet for supplying a mask pack sheet according to another embodiment of the present disclosure.

FIGS. 10A and 10B are views showing a device for manufacturing a user-customized home beauty mask pack according to another embodiment of the present disclosure.

FIGS. 11A and 11B are views showing a device for manufacturing a user-customized home beauty mask pack according to still another embodiment of the present disclosure.

### BEST MODE

Hereinafter, preferred embodiments of the present disclosure will be described in detail with reference to the attached drawings.

The embodiments of the present disclosure are provided to more fully explain the present disclosure to those skilled in the art, and the following embodiments may be modified in various other forms, and the scope of the present disclosure is not limited to the following embodiments. Rather, these embodiments are provided to more faithfully and completely convey the idea of the present disclosure to those skilled in the art.

In addition, in the drawings below, the thickness and size of each layer are exaggerated for convenience and clarity of explanation, and like symbols in the drawings represent like elements. As used herein, the term “and/or” includes any and all combinations of one or more of the listed items.

The terms used in this specification are used to describe particular embodiments and are not intended to limit the present disclosure. As used herein, the singular form may include the plural form unless the context clearly dictates otherwise. In addition, as used herein, the terms “comprise” and/or “comprising” specify the presence of stated features, numbers, steps, operations, members, elements and/or groups thereof, but do not preclude the presence or addition of one or more other features, numbers, operations, members, elements and/or groups thereof. The terms first, second, etc., may be used to describe various components, but the components should not be limited by these terms. These terms are used only to distinguish one component from another.

Hereinafter, the embodiments of the present disclosure will be described with reference to drawings schematically illustrating ideal embodiments of the present disclosure. In the drawings, for example, the size and shape of members may be exaggerated for convenience and clarity of explanation, and in actual implementation, variations in the depicted shapes may be expected. Therefore, the embodiments of the present disclosure should not be construed as being limited to the specific shapes of the members or regions illustrated in the present specification. Hereinafter, various embodiments of the present disclosure will be described with reference to the drawings.

The term “mask pack sheet” described below refers to a pure mask pack that is not impregnated with any functional raw materials, while the term “mask pack” refers to a mask pack sheet that has functional raw materials absorbed therein according to a mixing ratio.

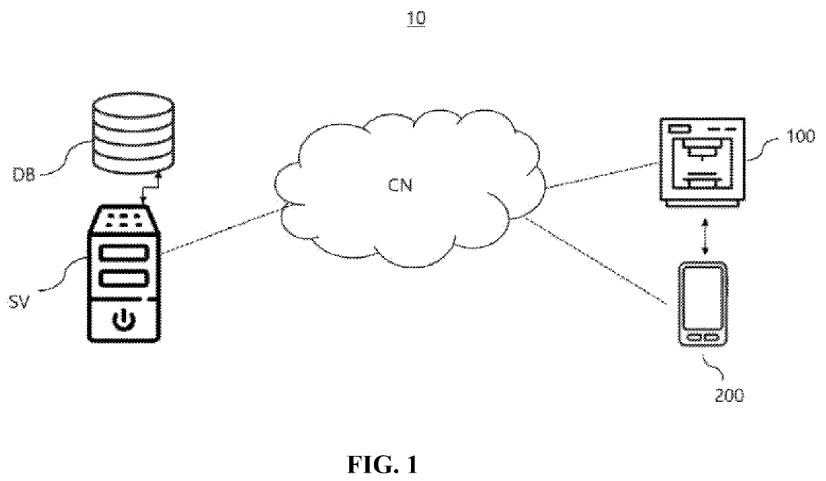

FIG. 1 is a configuration view of a system for manufacturing a user-customized home beauty mask pack according to an embodiment of the present disclosure.

Referring to FIG. 1, a system 10 for manufacturing a user-customized home beauty mask pack may include a database DB, an artificial intelligence server SV, a user terminal 100, and a device 200 for manufacturing a home beauty mask pack. The database DB, the artificial intelligence server SV, the user terminal 100, and the device 200 for manufacturing a home beauty mask pack may transmit and receive data to and from each other through a communication network CN. The communication network CN may be composed of a wired communication network, a mobile communication network, a wireless network, and a combination thereof. For example, the wired communication network includes a wired network based on IP (Internet Protocol). The mobile communication network includes a 5G network, an LTE (long term evolution) network, and a WCDMA (wideband code division multiple access) network. The wireless network includes various types of wireless networks such as a Wi-Fi network. However, the communication network CN is not limited to a specific technology, as the communication network CN can include not only the networks already built as described above, but also those developed in the future. The user terminal 100 may include at least one of a smartphone, a tablet personal computer, a mobile phone, a video phone, an e-book reader, a desktop personal computer, a laptop personal computer, a netbook computer, a workstation, a server, a personal digital assistant (PDA), a portable multimedia player (PMP), an MP3 player, a mobile medical device, and a wearable device (e.g., smart glasses, a head-mounted device (HMD)).

The database DB may include multiple storage devices and may store various types of data. In an embodiment of the present disclosure, the database DB may store information related to a user's skin diagnosis and data for artificial intelligence learning. The skin diagnosis related information may be generated by the user terminal 100 or an IoT beauty device (not shown), and the artificial intelligence learning data may collectively refer to data utilized for AI model learning such as machine learning and deep learning. The IoT beauty device (not shown) includes skin measurement sensors such as a pH measurement sensor, a moisture measurement sensor, and an oil measurement sensor, and may generate skin measurement signals of a user such as pH data, moisture data, and oil data. The IoT beauty device may transmit measured data to the user terminal 100 through the communication network CN. Alternatively, the IoT beauty device may be connected to the user terminal 100 through a connection terminal such as a USB port and provide measured data to the user terminal 100.

The artificial intelligence server SV accumulates data entered through the database DB on the AI platform as a time series, and may predict the user's skin diagnosis on the basis of the accumulated time series data and the skin diagnosis prediction model. In an embodiment, the skin diagnosis prediction for the user may be performed using unsupervised learning that finds patterns inherent in data using skin measurement information and skin input information as input values. In this case, machine learning can be defined as a methodology that gives computers the ability to learn on their own without being explicitly programmed, which is a way for programs to learn patterns in data on their own based on data. The machine learning is divided into supervised learning and unsupervised learning depending on whether the correct answer is specified in the data required for learning, and is divided, depending on the purpose of use, into: classification, which divides data into a finite number of categories; regression, which maps data to continuous values; clustering, which groups similar data; and dimension reduction, which maps multidimensional data to a representative lower dimensional space. Deep learning is a machine learning technique that is based on an artificial neural network (ANN) algorithm that mimics the synapse overlap in the human brain structure, and a deep learning architecture may include a deep neural network (DNN) with multiple hidden layers between the input layer and the output layer, a convolutional neural network (CNN) that places filters necessary for factor extraction in front of the hidden layer and learns the filters together, and a recurrent neural network (RNN) that can process time series data by stacking artificial neural networks for each time.

In an embodiment, the user terminal 100 may capture the user's face by means of a built-in camera, and the captured user face image may be utilized as skin diagnosis information. In addition, the user terminal 100 may input skin input information by means of an input means and provide the input information to the artificial intelligence server SV. The skin input information may be obtained through a social networking service (SNS), a skin diagnosis questionnaire, and a GPS receiver, and may include at least one of lifestyle information extracted from the user's SNS, information on an electronic skin diagnosis questionnaire filled out by the user, the user's location information received from the GPS receiver, the user's skin disease information and the user's genetic information issued by a specialized institution, and the humidity, temperature, fine dust, ultraviolet rays, and ozone index of the location where the user is from a site that provides weather information. The SNS may include any one of Facebook, Twitter, YouTube, Instagram, TikTok, Kakao Story, Pinterest, LinkedIn, Band, Kakao Group, Between, Pola, and Around. The lifestyle information may include at least one of the user's eating habits, water intake, outdoor activities, diet, pregnancy/childbirth, menstruation, lack of sleep, seasonal changes, and stress.

In an embodiment, the IoT beauty device (not shown) is linked to the user terminal 100 and may generate skin measurement information of the user. The skin measurement information is a value measured by means of various sensors and may include at least one of the degree of UV exposure for the user, oil and moisture content in the user's skin, skin temperature, skin pH, and skin thickness of the user. The user terminal 100 may receive the skin measurement information from the IoT beauty device (not shown) and visually display the received information to the user.

The database DB may store big data including learning data for predicting the user's skin diagnosis information from the skin measurement information and the skin input information. The artificial intelligence server SV may extract a specific pattern from the skin measurement information and the skin input information on the basis of the big data and predict the user's skin diagnosis information from the extracted specific pattern. The skin diagnosis information includes the skin's elasticity, moisture, pigmentation, anti-oxidation and sensitivity levels, the elasticity, moisture, pigmentation, anti-oxidation and sensitivity of the skin may be read from data including the user's pores, skin temperature, oil, moisture, skin tone, skin pH, skin thickness, wrinkles, pigmentation, blemishes, sebum and dead skin cells, and the data may be predicted from the first and second skin measurement information and the skin input information.

The device 200 for manufacturing a home beauty mask pack may receive the predicted skin diagnosis information, generate a control signal corresponding to the predicted skin diagnosis information, and manufacture a customized mask pack for the user based on the control signal. To be specific, the control signal may be used as a control signal to discharge one of the mask pack sheets stored in a stacked manner in the mask pack sheet supply unit described below, and may be used as a signal to control the ejection of various functional raw materials for mask packs, which will be described later, according to the mixing ratio. The mask pack sheet supply unit may store various types of mask pack sheets (e.g., T-zone, U-zone, V-zone, cheek pack, eye pack, etc.) separately by type.

The mask pack sheets stored in the mask pack sheet supply unit may be selected on the basis of the predicted skin diagnosis information and one of the sheets may be discharged to the space where functional raw materials are supplied, and functional raw materials may be dispersed or supplied inside and on the surface of the discharged mask pack sheet according to the predicted skin diagnosis information. The functional materials are effective for whitening, wrinkles, nutrition, moisturizing, allergy care, and exfoliation, and each ingredient may exist in liquid, powder, capsule, pill, or mixed form. In addition, the hydrating ingredients may include amino acids, ceramides, hyaluronic acid (sodium hyaluronate), or a mixture thereof, the ingredients for preventing skin dryness may include dipotassium glycyrrhizate, cica, or a mixture thereof, the whitening care ingredients may include arbutin, placenta, or a mixture thereof, and the ingredients for promoting skin elasticity may include collagen, retinol, or a mixture thereof. However, the present disclosure is not limited thereto.

In an embodiment, the device 200 for manufacturing a home beauty mask pack may be operated by voice recognition and, as an additional function, may have a cooling/heating function for cooling or warming a mask pack sheet in which the effective ingredients are dispersed or absorbed. For example, a warm mask pack can be used in cold weather like winter, while a cold mask pack can be used in hot weather, which may alleviate user discomfort.

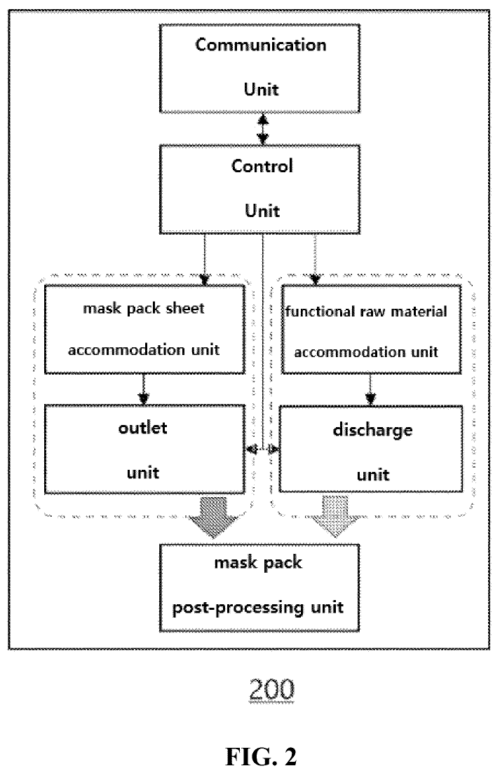

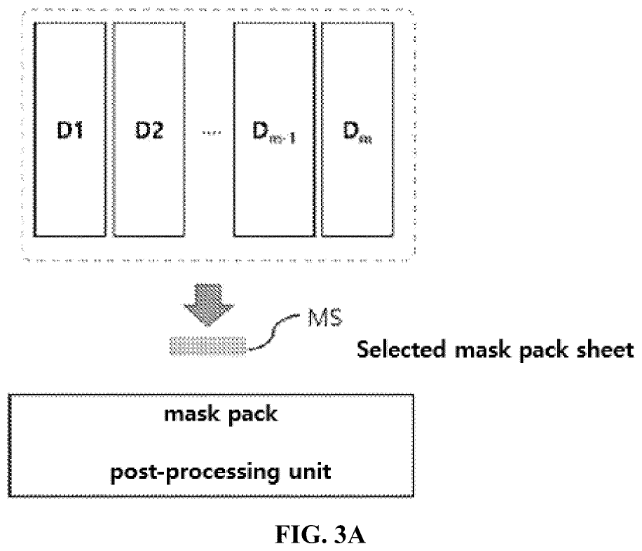

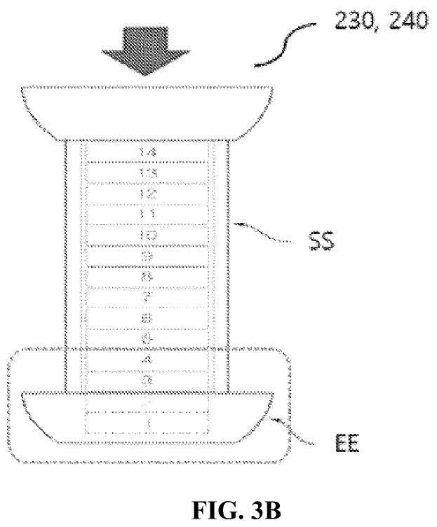

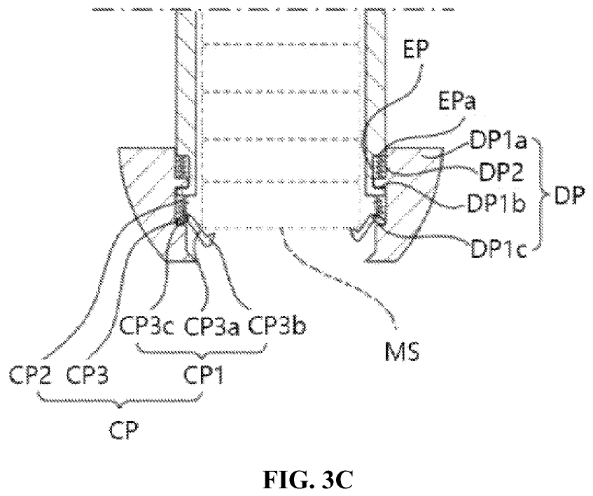

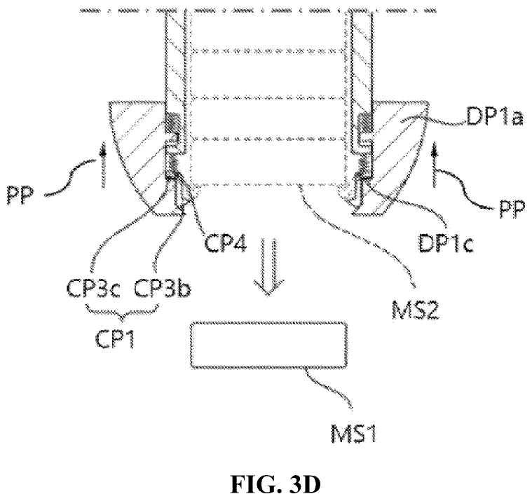

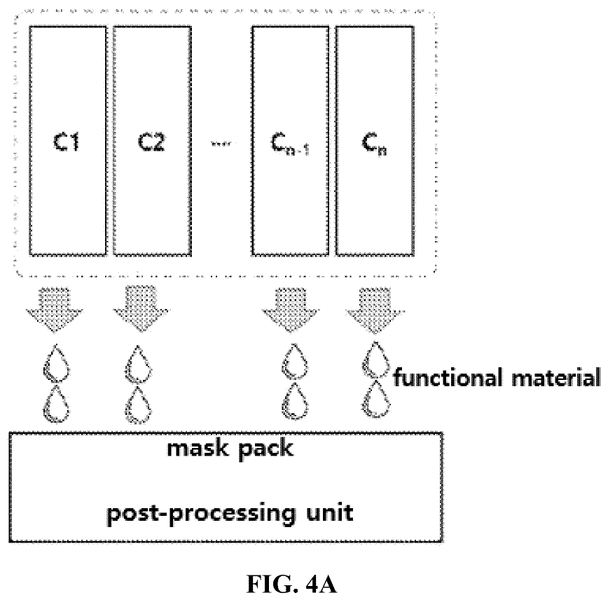

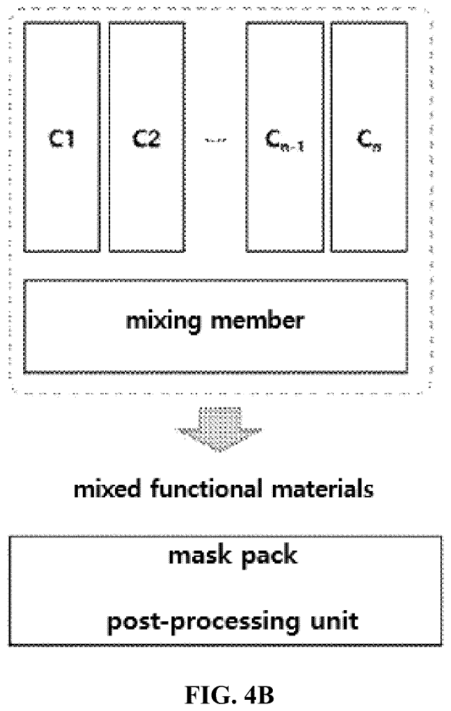

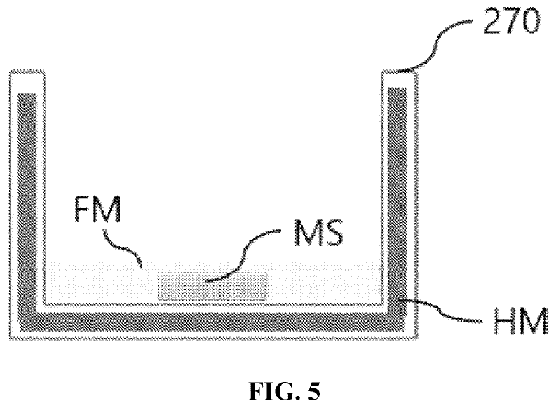

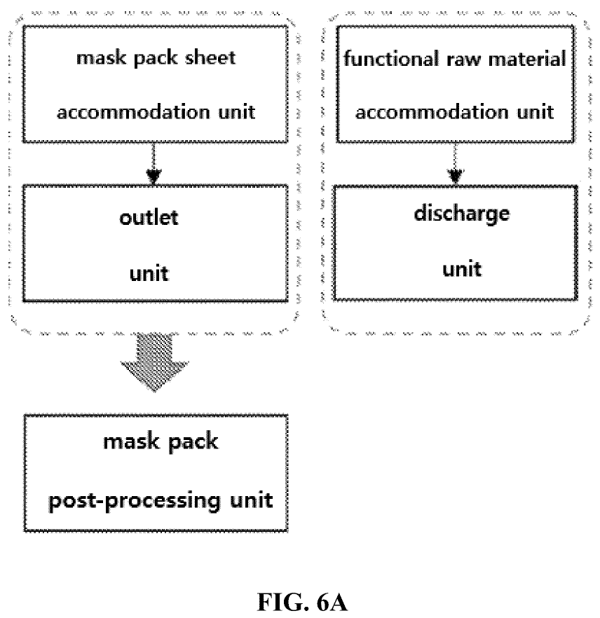

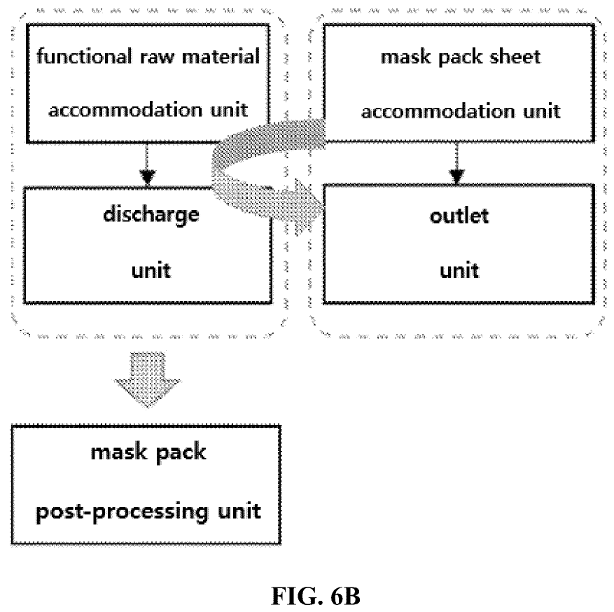

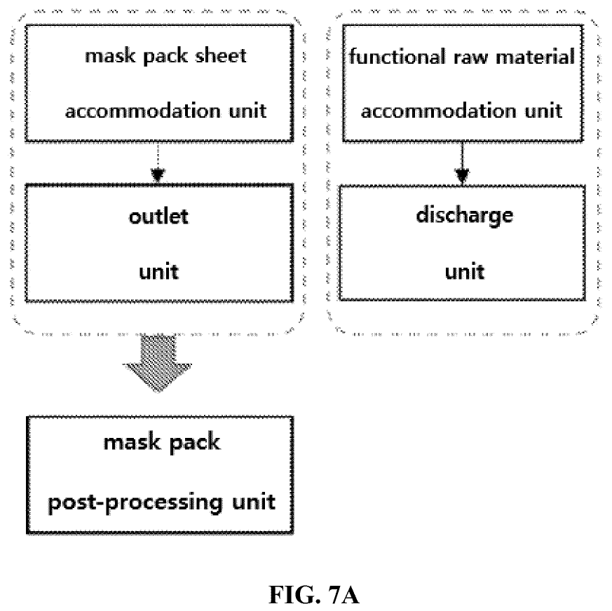

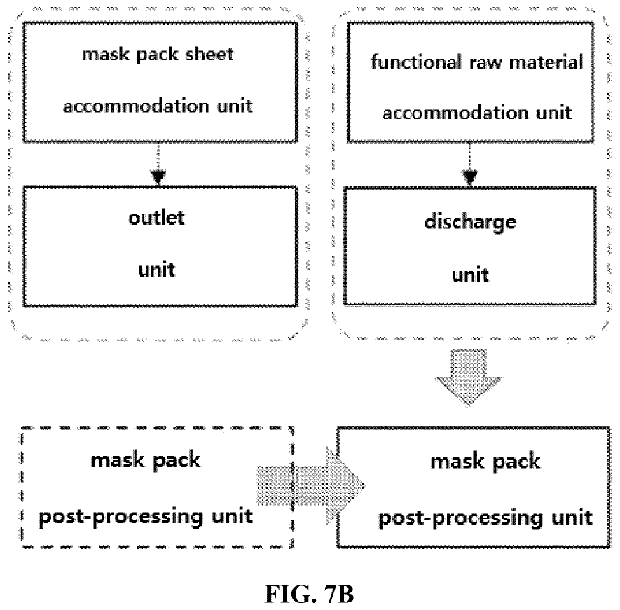

FIG. 2 is a configuration view of the device 200 for manufacturing a user-customized home beauty mask pack according to an embodiment of the present disclosure, FIGS. 3A to 3D are detailed views of a mask pack sheet supply unit according to an embodiment of the present disclosure, FIGS. 4A and 4D are detailed views of a functional raw material supply unit according to an embodiment of the present disclosure, FIG. 5 is a detailed view of a mask pack post-processing unit according to an embodiment of the present disclosure, and FIGS. 6A to 7B are views showing the discharge of a mask pack sheet and the ejection of a functional raw material according to the embodiments of the present disclosure.

Referring to FIG. 2, the device 200 for manufacturing a home beauty mask pack may include a communication unit 210, a control unit 220, a mask pack sheet supply unit MSS, an functional raw material supply unit FMS, and a mask pack post-processing unit 270. The mask pack sheet supply unit MSS may include a mask pack sheet accommodation unit 230 and a mask pack sheet outlet unit 240, and the functional raw material supply unit FMS may include a functional raw material accommodation unit 250 and a functional raw material discharge unit 260. The mask pack sheet supply unit MSS and the functional raw material supply unit FMS may be attached to and detached from the device, and the user may detach the mask pack sheet supply unit MSS and the functional raw material supply unit FMS to refill the mask pack sheets and functional raw materials. In addition, without limitation, the functional raw material supply unit FMS is a cartridge type that can be easily replaced by the user.

In an embodiment, the communication unit 210 may be configured as a communication module, such as a wired/wireless LAN card, a WCDMA modem, an LTE modem, or a 5G modem, and may provide skin input information and skin measurement information to a server through the communication network CN or receive skin diagnosis prediction information from the server. The communication unit 210 may provide the received skin diagnosis information or skin diagnosis prediction information to the control unit 220.

In an embodiment, the device 200 for manufacturing a home beauty mask pack may include: the mask pack sheet supply unit MSS for separately accommodating various types of mask pack sheets and including a mask pack sheet outlet port; the functional raw material supply unit FMS for separately accommodating a plurality of functional raw materials provided as a beauty and nutritional ingredient of a mask pack and including a functional raw material spout port; the control unit 220 for controlling the mask pack sheet supply unit MSS and the functional raw material supply unit FMS so that one of the mask pack sheets is discharged through the mask pack sheet outlet port and the functional raw materials are ejected through the functional raw material spout port according to a mixing ratio; and the mask pack post-processing unit 270 which includes an internal space open in a direction facing the discharge direction of the mask pack sheet outlet port and the ejection direction of the functional raw material spout port, and accommodates the discharged mask pack sheet and the ejected functional raw materials in the internal space so that the ejected functional raw materials are absorbed into the discharged mask pack sheet. The control unit 220 may receive user skin diagnosis information by being connected to the user terminal or at least one server SV through the communication unit 210 to determine one of the mask pack sheets and the mixing ratio of the functional raw materials, and may control the mask pack sheet outlet port so that the determined mask pack sheet is discharged from the mask pack sheet supply unit based on the user skin diagnosis information. In addition, the control unit 220 may control the functional raw material spout port so that the functional raw materials are ejected from the functional raw material supply unit FMS according to the determined mixing ratio.

Referring to FIG. 3A, the mask pack sheet supply unit MSS consisting of the mask pack sheet accommodation unit 230 and the mask pack sheet outlet unit 240 may include multiple units D1, D2, . . . , Dm according to the mask pack wearing area or mask pack shape. The shapes of mask packs include, for example, U-zone, T-zone, cheek pack, eye pack, eye wrinkle pack, neck pack, and face mask.

To be specific, the mask pack sheet supply unit D1 may accommodate U-zone shaped mask pack sheets in the mask pack sheet accommodation unit 230 and, under the control of the control unit 220, may discharge a U-zone shaped mask pack sheet place at the lowermost end of the mask pack sheet accommodation unit 230 through the mask pack sheet outlet unit 240.

Similarly, the mask pack sheet supply unit D2 may accommodate T-zone shaped mask pack sheets in the mask pack sheet accommodation unit 230 and, under the control of the control unit 220, may discharge a T-zone shaped mask pack sheet place at the lowermost end of the mask pack sheet accommodation unit 230 through the mask pack sheet outlet unit 240. The mask pack sheet supply unit D3 may accommodate nose zone shaped mask pack sheets, the mask pack sheet supply unit D4 may accommodate eye zone shaped mask pack sheets, the mask pack sheet supply unit D5 may accommodate eye wrinkle pack sheets, the mask pack sheet supply unit D6 may accommodate neck pack sheets, and the mask pack sheet supply unit D7 may accommodate face mask pack sheets, and each mask pack sheet supply unit may discharge a mask pack sheet through the mask pack sheet outlet unit 240 under the control of the control unit 220. However, the present disclosure is not limited to the above-mentioned shapes of mask packs, and other various shapes of mask packs may be applied.

In an embodiment, the mask pack sheet accommodation unit 230 may accommodate mask pack sheets in a stacked manner according to the type of mask pack. For example, in order to facilitate the discharge and storage of selective mask pack sheets, the mask pack sheets may be folded at least twice to have a predetermined thickness and stacked one by one inside the mask pack sheet accommodation unit 230. FIG. 3B shows an example in which 14 identical-shaped mask pack sheets are stacked. However, 14 or fewer or 14 or more identical-shaped mask sheets may be stacked in the internal space SS of the mask pack sheet accommodation unit 230.

The mask pack sheet outlet unit 240 may discharge the mask pack sheet at the lowermost mask pack sheet among the mask pack sheets stacked in in the mask pack sheet accommodation unit 230 to the mask pack post-processing unit 270 according to the control signal of the control unit 220. To be specific, the lower part EE of the mask pack sheet outlet unit 240 is configured to temporarily release a catch member so that only the lowermost mask pack sheet among the mask pack sheets stacked in the mask pack sheet accommodation unit 230 is discharged due to rotational movement or up-and-down movement according to the control signal of the control unit 220.

FIG. 3C shows a state before the lowermost mask pack sheet of the mask pack sheet accommodation unit 230 is discharged, and FIG. 3D shows a state in which the lowermost mask pack sheet of the mask pack sheet accommodation unit 230 is discharged based on the control signal of the control unit 220.

The mask pack sheet accommodation unit 230 accommodates a plurality of unit mask pack sheets by stacking the sheets, and the may be configured such that the lower surface thereof is open and the upper surface thereof is initially or eventually sealed by a separate sealing member that can be removed. Although in the embodiment of the present disclosure, the mask pack sheet accommodation unit 230 is illustrated as a cylindrical member with a cross-section formed in an annular shape, the present disclosure is not limited thereto, and may be implemented as accommodation units of various external shapes, such as a regular polyhedron, an oval, etc., and having an internal accommodation space.

Referring to FIG. 3C, the mask pack sheet outlet unit 240 may include an opening/closing guide means DP and an opening/closing means CP. The opening/closing guide means DP temporarily slides along the outer surface of the mask pack sheet accommodation unit SS in the longitudinal direction of the mask pack sheet accommodation unit SS by the control signal of the control unit 220 to link the opening/closing means CP to be described later.

As shown in FIG. 3C, the opening/closing guide means DP may include a sliding part DP1a and a first elastic member DP2. The sliding part DP1a may be slidably coupled to the lower outer surface of the mask pack sheet accommodation unit SS by the control signal of the control unit 220. In addition, the central portion of the sliding part DP1a is empty so that a unit mask pack sheet may be discharged, and the inner surface of the sliding part DP1a may be coupled to the outer surface of the accommodation unit SS so as to slide along the outer surface of the accommodation unit SS with a constant displacement. To this end, a guide groove EP is formed on the outer surface of the accommodation unit SS, and correspondingly, a guide protrusion DP2 that is inserted in the guide groove EP may be formed on the inner surface of the sliding part DP1a that comes into contact with the outer surface of the accommodation unit SS.

In an embodiment, the first elastic member DP2 has one end connected to the outer surface of the accommodation unit SS and the other end connected to the inner surface of the sliding part DP1a, and may provide a restoring force in the longitudinal direction of the accommodation unit SS. To be specific, one end of the first elastic member DP2 may be fixed to an upper side surface EPa of the guide groove EP, and the other end may be fixed to a side surface DP1c of the guide protrusion DP2.

The first elastic member DP2 may be configured as a compression spring, and when there is no external force on the sliding part DP1a, the first elastic member DP2 may press the sliding part DP1a downward (in the lower direction in FIG. 3C), and when the sliding part DP1a moves upward (in the upper direction in FIG. 3C) due to an external force, the first elastic member DP2 may provide a restoring force so that the sliding part DP1a moves downward again.

The opening/closing guide means DP having the above configuration has a structure in which, when the sliding part DP1a is moved upward under the control of the control unit 220, the opening/closing guide means DP retreats upward by the reaction force and temporarily slides along the outer surface of the accommodation unit SS and then is restored. The opening/closing guide means DP functions to discharge the lowermost unit mask pack sheet accommodated in the accommodation unit SS in conjunction with the opening/closing means CP described later.

In an embodiment, the opening/closing means CP serves to hold the lowermost unit mask pack sheet accommodated in the accommodation unit SS or release the sheet to be discharged. The opening/closing means CP may be provided at the lower portion of the accommodation unit SS so as to be linked with the above-described opening/closing guide means DP. Specifically, the opening/closing means CP may be configured to include a pair of latch parts CP1 and a second elastic member CP2.

The pair of latch parts CP1 is a member to hold the lowermost unit mask pack sheet or release the lowermost unit mask pack sheet from being held and to be discharged out in conjunction with the linear movement of the sliding part DP1a, and the latch parts CP1 may be rotatably coupled to the lower portion of the accommodation unit SS so as to face each other.

The latch part CP1 includes a load part CP3a, a holding part CP3b, and an extension part CP3c. The load part CP3a is a portion that forms the body of the latch part CP1 and is rotatably connected to the lower portion of the accommodation unit SS about a hinge axis CP3. The holding part CP3b may be formed by extending upwardly from one end of the load part CP3a and may hold the lowermost unit mask pack sheet. The extension part CP3c may be formed by extending downwardly from one end of the load part CP3a. In addition, a catch part DP1c is provided on the sliding part DP1a in correspondence with the extension part CP3c. The catch part DP1c is formed on the inner surface of the sliding part DP1a, and is structured to come into contact with the extension part CP3c when there is no striking motion. Due to this structure, when the sliding part DP1a moves in a straight line, the extension part CP3c rotates around the hinge axis CP3 in conjunction with the linear movement of the sliding part DP1a. The rotation of the extension part CP3c causes the rotation of the holding part CP3b, so that the pair of opposingly provided catch parts CP3b is temporarily separated.

Referring to FIG. 3D, in the accommodation unit of the present embodiment, mask pack sheets corresponding to 14 uses may be accommodated by dividing the sheets, folding the sheets individually, and then stacking the sheets. By the control signal of the control unit 220, the sliding part DP1a of the opening/closing guide means DP is temporarily pushed back (upward in FIG. 3D), and the latch part CP1 rotates counterclockwise around the hinge axis CP3 by the catch part DP1c formed on the sliding part DP1a. At this time, as the holding parts CP3b of the opposingly formed latch parts CP1 are mutually separated, the lowermost unit packaging content S1 that was being supported by the holding parts CP3b is released. As a result, the lowermost unit mask pack sheet MS1 may be discharged.

Afterwards, due to the restoring force of the first elastic member DP2, the sliding part DP1a moves forward (downward in FIG. 3D), and as the catch part DP1c releases the external force related to the rotation of the extension part CP3c, the latch part CP1 rotates clockwise around the hinge axis CP3 by the restoring force of the second elastic member CP2, thereby holding the next stacked unit mask pack sheet MS2.

Through the above process, the user may use the mask pack sheets one by one by discharging the sheets according to the control signal of the control unit 220 generated based on AI-based user skin diagnosis prediction information. In an embodiment, the control unit 220 may count the discharged mask pack sheets and provide the user with information about the number of unit mask pack sheets remaining in the accommodation unit SS, so that the user can fill the accommodation unit SS with mask pack sheets.

Referring to FIG. 4A, the functional raw material supply unit FMS including the functional raw material accommodation unit 250 and the functional raw material discharge unit 260 may be disposed in multiple numbers C1, C2, . . . , Cn corresponding to moisturizing ingredients, skin-drying prevention ingredients, whitening ingredients, and elasticity ingredients, respectively. The functional raw materials may include, but are not limited to, ingredients effective for whitening, wrinkles, nutrition, moisture, allergy care, and exfoliation.

To be specific, the functional raw material supply unit C1 stores whitening ingredients in the functional raw material accommodation unit 250 and may eject the whitening ingredients in the functional raw material accommodation unit 250 according to the mixing ratio through the discharge unit 260 under the control of the control unit 220.

Similarly, the functional raw material supply unit C2 stores ingredients that are effective in removing wrinkles, and may eject the ingredients that are effective in removing wrinkles stored in the accommodation unit 250 through the discharge unit 260 under the control of the control unit 220. The functional raw material supply unit C3 stores nourishing ingredients, the functional raw material supply unit C4 stores hydrating ingredients, the functional raw material supply unit C5 stores allergy care ingredients, and the functional raw material supply unit C6 stores ingredients that are effective for dead skin cells, and each may eject functional raw materials according to the mixing ratio through the discharge unit 260 under the control of the control unit 220. However, the present disclosure is not limited to the above-mentioned types of functional raw materials, and various types of functional raw materials may be further included.

In another embodiment, referring to FIG. 4B, before functional raw materials that are ejected from the functional raw material supply unit C1 to Cm according to the mixing ratio are supplied to the mask pack post-processing unit 270, the functional raw materials may be supplied to a mixing member 265 disposed between the functional raw material supply unit C1 to Cm and the mask pack post-processing unit 270, and the functional raw materials may be rotated or rotated up and down so as to be evenly dispersed and mixed inside the mixing member 265.

Referring to FIG. 5A, the mask pack post-processing unit 270 may include a Peltier thermoelectric element HM for cooling or heating a mask pack sheet MS and functional raw materials FM in the internal space through a heat absorption process or a heat generation process according to a control signal of the control unit 220. Thus, in cold weather, the mask pack sheet and functional raw materials are heated to prevent the user from feeling cold, and in hot weather, the mask pack sheet MS and functional raw materials FM are cooled to reduce the user's discomfort and feeling of rejection when wearing the mask pack. That is, the mask pack can be worn cold or hot depending on the user's preference. The Peltier thermoelectric element HM may be placed on the inner surface, outer surface of the mask pack post-processing unit 270, or may be inserted into the mask pack post-processing unit 270.

The control unit 220 may control the first polarity of the power input from a power source (not shown) so as to be applied to the Peltier thermoelectric element HM, thereby allowing heat absorption in the Peltier thermoelectric element HM. In addition, the control unit 220 may reversely control the polarity (second polarity) of the power input from the power source (not shown) so as to be applied to the Peltier thermoelectric element HM, thereby allowing heat generation in the Peltier thermoelectric element HM.

In still another embodiment, the mask pack post-processing unit 270 may be composed of a main body formed of a thermally conductive resin, an insert metal part joined to the outside of the main body, and a thermoelectric module in contact with the insert metal part in order to cool or heat the mask pack sheet MS and the functional raw materials FM. The main body is used as a passage through which heat is transferred by conduction between the thermoelectric module and the accommodation unit 122, and it is preferable that the main body be made of a thermally conductive plastic resin to enhance the heat conduction effect. In addition, the main body is designed to have a large area in contact with the thermoelectric module to increase the heat conduction effect, and the wall of the main body 120 along the path through which heat is transferred is designed to be as thin as possible. The insert metal part may be installed so as to be coupled to the outside of the main body and to be in contact with the thermoelectric module. The insert metal part may be made of aluminum having excellent thermal conductivity to effectively transfer heat or cold generated from the thermoelectric module. The thermoelectric module may be installed on one side of the main body as a temperature-controllable device to perform a refrigeration or heating function according to the user's needs.

In an embodiment, in order to sequentially store mask pack sheets MS and functional raw materials FM into the internal space of the mask pack post-processing unit 270, the mask pack post-processing unit 270 may move left and right by a separate transfer member (not shown) as shown in FIGS. 6A and 6B, or the mask pack supply unit 230 and 240 and the functional raw material supply unit 250 and 260 may be rotated and moved without moving the mask pack post-processing unit 270 as shown in FIGS. 7A and 7B.

A rotary member (not shown) may be further included. The rotary member causes, at a first time point, the mask pack supply unit 230 and 240 to perform a first rotation so that the discharge direction of a mask pack sheet of the mask pack sheet outlet unit 240 and the opening direction of the mask pack post-processing unit 270 are aligned and the mask pack sheet is discharged as shown in FIG. 6A, and causes, at a second time point, different from the first time point, the mask pack post-processing unit 270 to perform a second rotation so that the opening direction of the mask pack post-processing unit 270 and the ejection direction of functional raw materials of the functional raw material discharge unit 240 are aligned and the functional raw materials are ejected as shown in FIG. 6B.

To be specific, due to the second rotation, the arrangement positions of the mask pack supply unit 230 and 240 and the functional raw material supply unit 250 and 260 may be switched with each other. For example, the functional raw material supply unit 250 and 260 may be positioned at the position of the mask pack supply unit 230 and 240 whereas the mask pack supply unit 230 and 240 may be positioned at the position of the functional raw material supply unit 250 and 260.

In another embodiment, the transfer member (not shown) may be further included. The transfer member places, at a first time point, the mask pack post-processing unit at a first position so that the discharge direction of a mask pack sheet of the outlet unit and the opening direction of the mask pack post-processing unit are aligned as shown in FIG. 7A, and transfers, at a second time point which is different from the first time point, the mask pack post-processing unit from the first position to a second position so that the opening direction of the mask pack post-processing unit and the ejection direction of functional raw materials of the discharge unit are aligned as shown in FIG. 7B.

In an embodiment, the control unit 220 may provide the user with information about the shape of a mask pack sheet to be discharged and the functional raw materials FM to be absorbed into the mask pack sheet MS before discharging the mask pack sheet MS into the internal space of the mask pack post-processing unit 270 or before ejecting the functional raw materials FM into the internal space of the mask pack post-processing unit 270. This can minimize matching errors by showing the above information to the user before manufacturing a mask pack, as the user's preference and skin diagnosis prediction results may differ.

In an embodiment, in response to a response signal from the user, the control unit 220 may correct the shape of a mask pack sheet and the mixing ratio of functional raw materials to be absorbed into the mask pack sheet, and may control to discharge and eject the mask pack sheet and the functional raw materials into the internal space of the mask pack post-processing unit according to the corrected shape of the mask pack sheet and the mixing ratio of the functional raw materials. For example, the user may send a response signal to the control unit 220 to ensure that the mask pack contains nutritional ingredients rather than moisturizing ingredients.

Meanwhile, the device 200 for manufacturing a user-customized home beauty mask pack may manufacture a customized mask pack with necessary ingredients for each needed skin area based on artificial intelligence by diagnosing, by means of a skin diagnosis app of a smart beauty device that operates through an authentication procedure, the user's skin condition, measuring the amount of necessary oil and moisture, and other necessary ingredients required by the user, and supplying raw materials containing ingredients necessary for the user's skin. For example, when the user's skin is very dry around the eyes, oily around the forehead, and dry around the mouth and cheeks, the user may make a mask pack with ingredients suitable for each skin type: eye patches for very dry skin, forehead patches for oily skin, and other patches for dry skin.

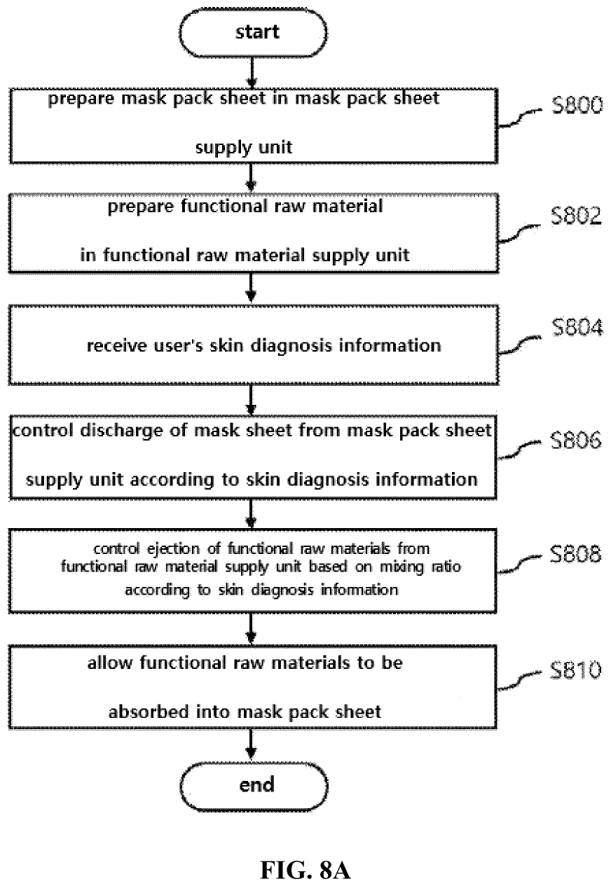

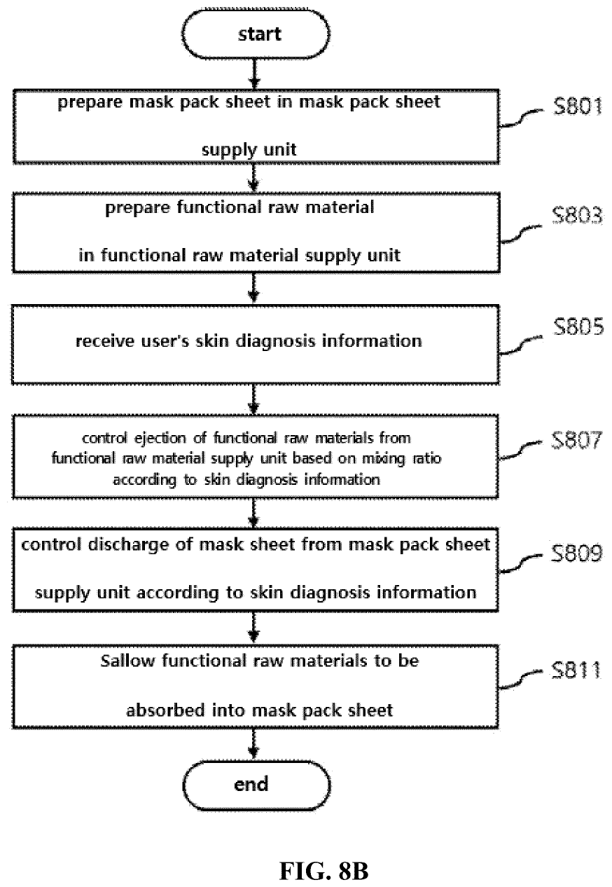

FIGS. 8A and 8B are flowcharts for showing a method for manufacturing a user-customized home beauty mask pack according to an embodiment of the present disclosure.

Referring to FIG. 8A, a method for manufacturing a user-customized home beauty mask pack may include: a step (800) of separately accommodating various types of mask pack sheets in a mask pack sheet supply unit including a mask pack sheet outlet port; a step (S802) of separately accommodating a plurality of functional raw materials provided as a beauty and nutritional ingredient of a mask pack in a raw material supply unit including a functional raw material spout port; a step (S804) of receiving skin diagnosis information from an external server or a user terminal; a step (S806) of discharging one of the mask pack sheets to the mask pack sheet supply unit through the mask pack sheet outlet port according to a control signal corresponding to the skin diagnosis information; a step (S808) of controlling the raw material supply unit so that the functional raw materials are ejected according to the mixing ratio through the functional raw material spout port; and a step (S810) of discharging the mask pack sheet into the internal space of a mask pack post-processing unit that is open in a direction facing the discharge direction of the mask pack sheet outlet port and the ejection direction of the functional raw material spout port, and ejecting functional raw materials onto the mask pack sheet according to the mixing ratio.

In another embodiment, as shown in FIG. 8B, functional raw materials may be ejected before the mask pack sheet. Since step S801 of FIG. 8B is identical to step S800 of FIG. 8A, step S803 of FIG. 8B is identical to step S802 of F FIG. 8A, step S805 of FIG. 8B is identical to step S804 of FIG. 8A, step S807 of FIG. 8B is identical to step S808 of FIG. 8A, step S809 of FIG. 8B is identical to step S806 of FIG. 8A, and step S811 of FIG. 8B is identical to step S810 of FIG. 8A, unless there is a contradiction, reference may be made to the description of FIG. 8A.

Although the aforementioned FIGS. 2, 6A to 7B show a structure in which the device 100 for manufacturing a user-customized home beauty mask pack has the mask pack sheet supply unit 230 and 240 that separately accommodates separate mask pack sheets, the present disclosure is not limited thereto and a separate capsule-type mask pack sheet may be used instead of the mask pack sheet supply unit 230 and 240. The capsule-type mask pack sheet may refer to a product that is stored and sold in individual containers before functional raw materials are injected. Optionally, the capsule-type mask pack may be stored by containing only basic ingredients such as moisture. Various types of mask pack sheets may be supplied in the form of individual capsule-type mask packs.

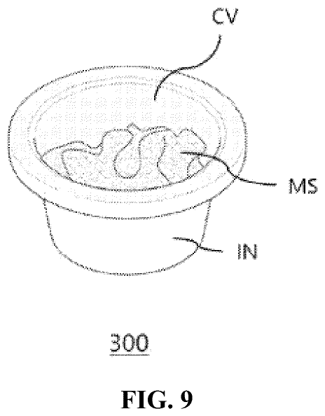

FIG. 9 is an example of a capsule-type mask pack sheet for supplying a mask pack sheet according to another embodiment of the present disclosure.

Referring to FIG. 9, a capsule-type mask pack product 300 may be composed of a mask pack sheet MS, a container IN that accommodates the mask pack sheet MS, and a cover sheet CV that seals the container IN. The mask pack sheet MS may be stored in the container in various forms. For example, since the mask pack sheet MS does not maintain a certain shape, the mask pack sheet MS may be placed in the container like a crumpled toilet paper, and the mask pack sheet MS may be folded in half or quarters to be placed into the container IN. Since the mask pack sheet MS may be transformed into various shapes depending on the shape of the container IN, various types of capsule-type mask pack products 300 may be provided. The container IN for putting the mask pack sheet MS may be made any one of glass, aluminum, magnetic, plastic, acrylic, paper vinyl, PET/PP container without limitation. A glass container that allows a user to visually check the mask pack sheet MS may be used, an aluminum packaging container may be used, a ceramic container may be used for aesthetic effects, a flexible plastic container may be used, or an acrylic container may be used. The shape of the container may be freely modified and used regardless of the material of the container mentioned. The cover sheet CV may be attached to the flange of the container IN to seal the container IN airtightly so that the mask pack MS stored in the container IN is not exposed to the outside air. The cover sheet CV may be made of an aluminum film or a plastic film that can be punctured by a puncture nozzle described later. When using the capsule-type mask pack product 300, the inflow of contaminants from the outside into the mask pack may be minimized, and thus the mask pack may be stored and used for a long period of time without being contaminated.

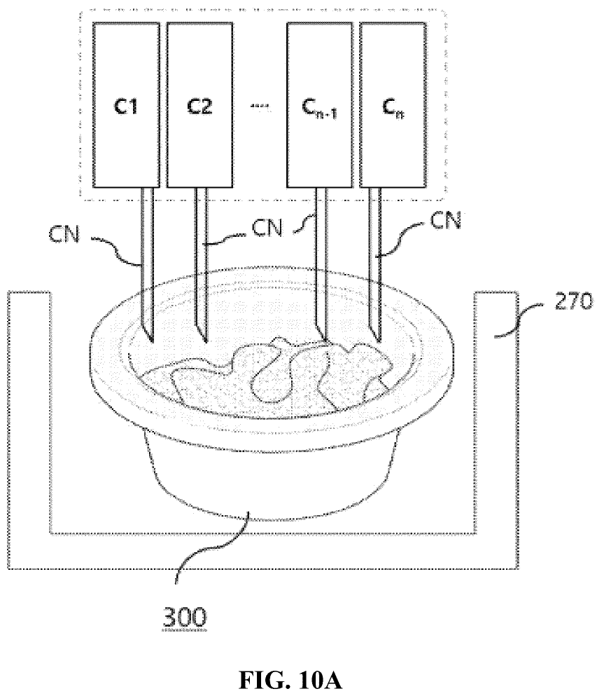

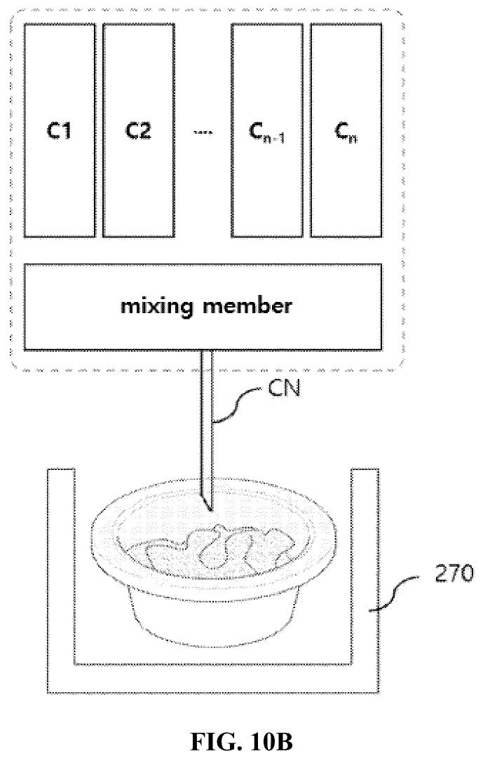

FIGS. 10A and 10B are views showing a device 200′ for manufacturing a user-customized home beauty mask pack according to another embodiment of the present disclosure. The device 200′ for manufacturing a user-customized home beauty mask pack may be composed of the communication unit 210, the control unit 220, the functional raw material supply unit FMS, and the mask pack post-processing unit 270 of the device 200 for manufacturing a user-customized home beauty mask pack excluding the mask pack sheet supply unit MSS described in FIG. 2. Instead of the mask pack sheet supply unit MSS, the capsule-type mask pack product 300 of FIG. 9 described above may be used.

Referring to FIG. 10A, a user first selects a desired capsule-type mask pack product 300, places the selected capsule-type mask pack product 300 in the mask pack post-processing unit 270, and lowers the functional raw material supply unit FMS under the control of the control unit 220 so that the cover sheet CV of the capsule-type mask pack product 300 is punctured. Thereafter, under the control of the control unit 220, the functional raw material supply unit FMS may eject functional raw materials according to a user-customized mixing ratio. The functional raw material supply unit FMS may be composed of cartridges C1, C2, . . . , Cn_1, Cn_2 storing functional raw materials and a plurality of puncture nozzles CN integrally connected to the respective cartridges. By integrating the cartridge and the puncture nozzle, functional raw materials may be hygienically injected into the container IN of the capsule-type mask pack product 300. One end of the puncture nozzle CN is pointed and sharp to easily puncture the cover sheet CV of the capsule-type mask pack product 300. At this time, one end of the puncture nozzle CN is positioned inside the container IN. When functional raw materials with the user-customized mixing ratio is injected into the container IN of the capsule-type mask pack product 300, the user may remove the cover sheet CV after a predetermined time to take out and use the mask pack MS in which the functional raw materials have been absorbed from the container IN.

Referring to FIG. 10B, before a plurality of functional raw materials is supplied from the functional raw material supply unit C1 to Cm according to the mixing ratio to the mask pack post-processing unit 270, by rotating the functional raw materials inside the mixing member 265 so that the functional raw materials are evenly dispersed and mixed, and puncturing the cover sheet CV of the capsule-type mask pack product 300 by means of the single puncture nozzle CN integrally connected to the mixing member 265, the mixed functional raw materials may be injected into the container IN. At this time, the cartridges C1, C2, etc., for accommodating functional raw materials, the mixing member 265, and the single puncture nozzle CN may be configured as an integral body, and each cartridge may be attached or detached from the mixing member 265.

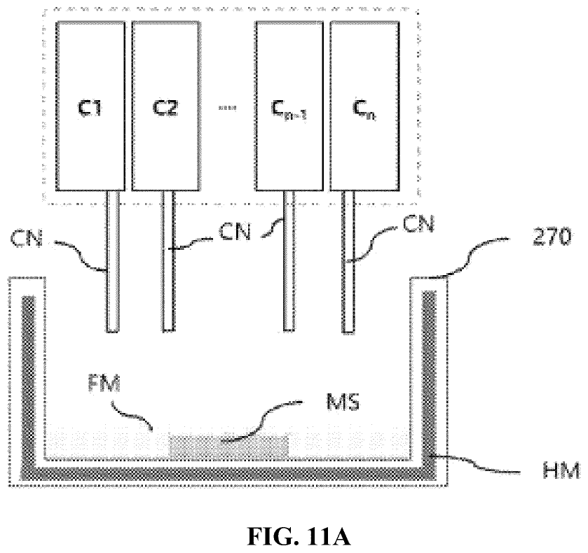

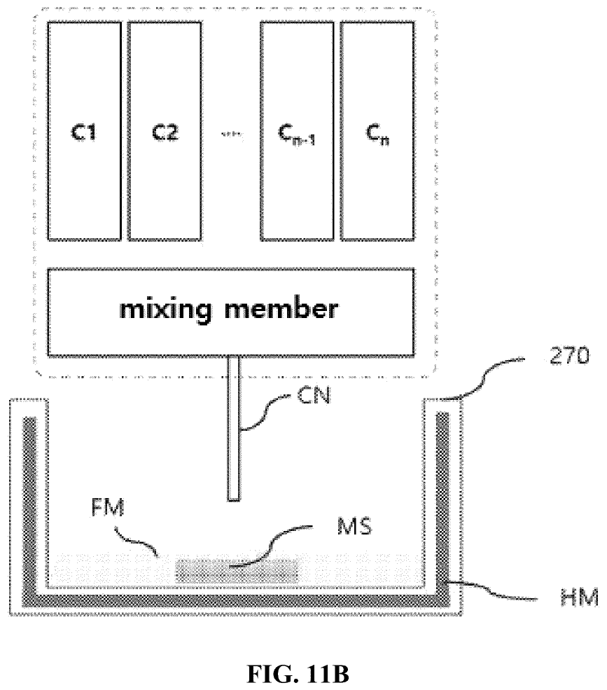

In still another embodiment, instead of using the capsule-type mask pack product 300, a mask pack product in general packaging may be used. In this case, the nozzle does not need a puncturing function, and the user may use a mask pack in general packaging by directly inserting the mask pack into the internal space of the mask pack post-processing unit 270 of FIG. 5 as described above, as shown in FIG. 11A and FIG. 11B.

FIGS. 11A and 11B are views showing a device for manufacturing a user-customized home beauty mask pack according to still another embodiment of the present disclosure.

Referring to FIG. 11A, the user may remove the packaging from the packaged mask pack product, place the mask pack MS with packaging removed into the internal space of the mask pack post-processing unit 270, and inject functional raw materials through individual general nozzles CN. At this time, as in FIG. 10A, the general nozzle may be configured as an integral part with the cartridge that accommodates a functional raw material, so that the user may easily and hygienically replace and use the cartridge.

Referring to FIG. 11B, the user may remove the packaging from the packaged mask pack product, place the mask pack MS with packaging removed into the internal space of the mask pack post-processing unit 270, and after a plurality of functional raw materials are ejected from the functional raw material supply unit C1 to Cm according to the mixing ratio, evenly dispersed, and mixed inside the mixing member 265 as in FIG. 10b, may inject mixed functional raw materials into the internal space of the mask pack post-processing unit 270 through the single nozzle CN. At this time, the cartridges C1, C2, etc., for accommodating functional raw materials, the mixing member 265, and the single nozzle CN may be configured as an integral body, and each cartridge may be attached or detached from the mixing member 265. In the case where there is no functional raw material left in the cartridge C1, the cartridge C1 may be replaced or the existing cartridge C1 may be used by filling the cartridge C1 with a functional raw material.

As described above, the present disclosure enables precise skin diagnosis by utilizing data measured from various IoT skin measurement devices together without relying on user input such as a questionnaire and a selfie taken by a user for skin diagnosis. In addition, in line with the recent trend of pursuing a free lifestyle at home, the device of the present disclosure may be linked to a smartphone app and allow a user to create and use a variety of mask packs tailored to their skin type based on skin measurement results and artificial intelligence algorithms, thereby increasing user accessibility, efficiency, and convenience.

Furthermore, the present disclosure enables the provision of effective ingredients necessary for the skin in a personalized manner in real time through continuous skin diagnosis analysis, enables the optimal mixing of necessary ingredients for each facial area (T-zone, U-zone, eye, cheek, etc.) and the production of patches with customized shapes for respective areas (eye, cheek, forehead, neck, and face) at home, and provides a scientific skin care solution that resolves concerns related to skin changes and future skin concerns through skin analysis history.

In addition, the present disclosure enables precise diagnosis of what nutrients are needed for each area of the user's face and to continuously monitor and manage the user's skin condition, and goes beyond simply manufacturing a mask pack and allows a user to manufacture and use a mask pack at home by area according to the individual's skin condition, weather, humidity, season, region of residence, personal schedule, and taste, thereby achieving simplicity, convenience, cost-effectiveness, and efficiency.

It will be apparent to those skilled in the art that the present disclosure described above is not limited to the above-described embodiments and the attached drawings, and that various substitutions, modifications, and changes are possible within the scope that does not depart from the technical spirit of the present disclosure.

### Industrial Applicability

An embodiment of the present disclosure may be applied to a device and method for manufacturing a user-customized home beauty mask pack.

1. A device for manufacturing a user-customized home beauty mask pack, the device comprising:
a mask pack sheet supply unit configured to separately accommodate various types of mask pack sheets and include a mask pack sheet outlet port;
a functional raw material supply unit configured to separately accommodate a plurality of functional raw materials provided as a beauty and nutritional ingredient of a mask pack and include a functional raw material spout port;
a control unit configured to control the mask pack sheet supply unit and the functional raw material supply unit so that one of the mask pack sheets is discharged through the mask pack sheet outlet port and the functional raw materials are ejected through the functional raw material spout port according to a mixing ratio; and
a mask pack post-processing unit configured to include an internal space open in a direction facing a discharge direction of the mask pack sheet outlet port and an ejection direction of the functional raw material spout port, and accommodate the discharged mask pack sheet and the ejected functional raw materials in the internal space so that the ejected functional raw materials are absorbed into the discharged mask pack sheet.

2. The device of claim 1, wherein the control unit receives user skin diagnosis information by being connected to a user terminal or at least one server through a communication unit to determine the one of the mask pack sheets and the mixing ratio of the functional raw materials, controls the mask pack sheet outlet port so that the determined mask pack sheet is discharged from the mask pack sheet supply unit based on the user skin diagnosis information, and controls the functional raw material spout port so that the functional raw materials are ejected from the functional raw material supply unit according to the determined mixing ratio.

3. The device of claim 1, wherein the mask pack sheet supply unit comprises:
at least one mask pack sheet accommodation unit configured to accommodate mask pack sheets in a stacked manner according to a type of mask pack; and
at least one mask pack sheet outlet unit configured to discharge, according to a control signal of the control unit, a lowermost mask pack sheet among mask pack sheets stacked in a corresponding mask pack sheet accommodation unit to the mask pack post-processing unit.

4. The device of claim 1, wherein the functional raw material supply unit comprises:
at least one functional raw material accommodation unit configured to accommodate functional raw materials respectively corresponding to a moisturizing ingredient, a skin-drying prevention ingredient, a whitening ingredient, and an elasticity ingredient; and
at least one functional raw material discharge unit configured to eject, according to a control signal of the control unit, the functional raw materials accommodated in the functional raw material accommodation unit to the mask pack post-processing unit.

5. The device of claim 4, wherein the functional raw material supply unit further comprises:
a mixing member configured to mix a plurality of functional raw materials supplied according to the mixing ratio from the functional raw material accommodation unit and deliver the mixed functional raw materials to the functional raw material discharge unit.

6. The device of claim 1, wherein the mask pack post-processing unit comprises:
a Peltier thermoelectric element for cooling or heating a mask pack sheet and functional raw materials in the internal space through a heat absorption process or a heat generation process according to a control signal of the control unit.

7. The device of claim 1, further comprising:
a rotary member configured to rotate, at a first time point, the mask pack sheet supply unit so that the discharge direction of a mask pack sheet of the mask pack sheet outlet port and the opening direction of the mask pack post-processing unit are aligned, and
rotate, at a second time point, different from the first time point, the mask pack post-processing unit so that the opening direction of the mask pack post-processing unit and the ejection direction of functional raw materials of the functional raw material discharge unit are aligned.

8. The device of claim 1, further comprising:
a transfer member configured to place, at a first time point, the mask pack post-processing unit in a first position so that the discharge direction of a mask pack sheet of the mask pack sheet outlet port and the opening direction of the mask pack post-processing unit are aligned, and
transfer, at a second time point, different from the first time point, the mask pack post-processing unit from the first position to a second position so that the opening direction of the mask pack post-processing unit and the ejection direction of functional raw materials of the functional raw material discharge unit are aligned.

9. The device of claim 1, wherein the control unit provides a user with information about a shape of the mask pack sheet to be discharged and the functional raw materials to be absorbed into the mask pack sheet before discharging the mask pack sheet into the internal space of the mask pack post-processing unit or before ejecting the functional raw materials into the internal space of the mask pack post-processing unit.

10. The device of claim 9, wherein the control unit corrects the shape of the mask pack sheet and a mixing ratio of the functional raw materials to be absorbed into the mask pack sheet in response to a response signal from the user, and controls a discharge and ejection of the mask pack sheet and the functional raw materials into the internal space of the mask pack post-processing unit according to the corrected shape of the mask pack sheet and the mixing ratio of the functional raw materials.

11. The device of claim 1, wherein the types of the mask pack sheets include any one of the following: U-zone, T-zone, nose pack, eye pack, eye wrinkle pack, neck pack, and face mask.

12. A method for manufacturing a user-customized home beauty mask pack, the method comprising:
separately accommodating various types of mask pack sheets in a mask pack sheet supply unit including a mask pack sheet outlet port;
separately accommodating a plurality of functional raw materials provided as a beauty and nutritional ingredient of a mask pack in a raw material supply unit including a functional raw material spout port;
discharging one of the mask pack sheets to the mask pack sheet supply unit through the mask pack sheet outlet port according to a control signal from a control unit;
controlling the raw material supply unit so that the functional raw materials are ejected according to a mixing ratio through the functional raw material spout port; and
discharging the one of the mask pack sheets into an internal space of a mask pack post-processing unit that is open in a direction facing a discharge direction of the mask pack sheet outlet port and an ejection direction of the functional raw material spout port, and ejecting functional raw materials onto the mask pack sheet according to the mixing ratio.

13. The method of claim 12, further comprising:
receiving user skin diagnosis information by being connected to a user terminal or at least one server through a communication unit to determine the one of the mask pack sheets and the mixing ratio of the functional raw materials, controlling the mask pack sheet outlet port so that the determined mask pack sheet is discharged from the mask pack sheet supply unit based on the user skin diagnosis information, and controlling the functional raw material spout port so that the functional raw materials are ejected from the raw material supply unit according to the determined mixing ratio.

14. The method of claim 12, wherein the skin diagnosis information includes skin's elasticity, moisture, pigmentation, anti-oxidation, and sensitivity levels,
the skin's elasticity, moisture, pigmentation, anti-oxidation, and sensitivity levels are predicted from data including user's pores, skin temperature, oil, moisture, skin tone, skin pH, skin thickness, wrinkles, pigmentation, blemishes, sebum, and dead skin cells,
the skin diagnosis information further includes skin measurement information, and
the skin measurement information includes at least one of a degree of UV exposure for a user, oil and moisture content in user's skin, skin temperature, skin pH, and skin thickness of the user.

15. A device for manufacturing a user-customized home beauty mask pack, the device comprising:
a mask pack post-processing unit configured to place a capsule-type mask pack product into an internal space thereof, wherein the capsule-type mask pack product consists of a mask pack sheet, a container for accommodating the mask pack sheet, and a cover sheet for sealing the container;
a functional raw material supply unit including at least one cartridge configured to separately accommodate a plurality of functional raw materials provided as a beauty and nutritional ingredient of a mask pack, and at least one puncture nozzle integrally connected with the cartridge; and
a control unit configured to lower the puncture nozzle integrally connected with the cartridge to puncture the cover sheet of the capsule-type mask pack product, control an end of the puncture nozzle to enter the container of the capsule-type mask pack product, and control an injection of the functional raw materials into the container of the capsule-type mask pack product according to a mixing ratio so that the functional raw materials are absorbed into the mask pack sheet.
